(19) United States
(12) Patent Application Publication
(10) Pub. No.: US 2025/0032520 A1
(43) Pub. Date: Jan. 30, 2025

(54) ORAL CANNABINOID COMPOSITIONS AND METHODS FOR TREATING NEUROLOGICAL DISEASES AND DISORDERS

(71) Applicant: Avicanna Inc., Toronto (CA)

(72) Inventors: Frantz Henri Emmanuel LE DEVEDEC, North York (CA); Dong AN, Markham (CA); Aras AZADIAN, Toronto (CA)

(73) Assignee: Avicanna Inc., Toronto, ON (CA)

(21) Appl. No.: 18/716,049

(22) PCT Filed: Dec. 1, 2022
(86) PCT No.: PCT/IB2022/061666

§ 371 Date: Jun. 3, 2024

Related U.S. Application Data
(60) Provisional application No. 63/264,903, filed on Dec. 3, 2021.

(51) Int. Cl. A61K 31/00; A61K 9/00; A61K 9/20; A61K 47/02; A61K 47/10; A61K 47/12; A61K 47/44

(52) U.S. Cl. CPC A61K 31/658; A61K 9/0053; A61K 9/2009; A61K 9/2013; A61K 9/2027; A61K 9/2031; A61K 9/2054; A61K 47/02; A61K 47/10; A61K 47/12; A61K 47/44

(57) ABSTRACT

Self-emulsifying drug delivery compositions are provided for oral delivery of cannabinoids. The cannabinoids are dissolved in a solubilizer together with at least one stabilizer to improve dissolution and stability. The cannabinoids are also gelled in a standardized matrix to control the release upon contact with aqueous media. Also provided are methods comprising uses of oral cannabinoid compositions for the treatment of a neurological disease and/or disorder in a subject.

### FIELD

The present disclosure relates to compositions for oral administration of one or more cannabinoids to patients and, more particularly, through self-emulsifying drug delivery systems (SEDDS).

### BACKGROUND

Cannabis has long been used for medicinal and recreational purposes. The active components of cannabis, i.e., phytocannabinoids, are effective as a drug against nausea and vomiting which are common side-effects of anaesthetics, opioids analgesics, chemotherapy for cancer and highly active anti-retroviral therapy (HAART for HIV-AIDS). Cannabis has also been used for a long period of time as a drug for relieving chronic neurogenic/neuropathic pain that is caused by surgical operations and by several disorders. Other medical indications include multiple sclerosis, depression, migraine, fibromyalgia, Parkinson syndrome and Gilles de la Tourette syndrome. It is also useful as spasmolytic, appetite stimulating, palliative and anti-convulsant medication.

Smoking is the most prevalent mode of use of cannabis. Smoking is a less desirable mode of administration for drugs, including medical cannabis since it has adverse effects on the lungs. Cannabis smoke carries more tar and other particulate matter as compared to tobacco and may be a cause of lung diseases including chronic obstructive pulmonary disease (COPD) and lung cancer. Furthermore, many patients find the act of smoking unappealing due to social constraints.

Another method frequently employed is vaporisation wherein the herb is heated to about 180° C., rather than burned so that harmful side-products are hardly formed. Additionally, the vapour may be cooled or further purified if desired before inhalation. Furthermore, the dosage is easily controlled by the patient since inhalation provides for a rapid onset and a fast delivery into the bloodstream. However, the use of a vaporiser is also not always convenient since it requires a place or spot where the patient can set up and use his or her vaporiser to undergo treatment. In this respect it is also time-consuming.

Oral administration is the easiest and most convenient route for non-invasive drug administration. However, due to the highly lipophilic nature of cannabinoids, they are soluble in lipids and some organic solvents while being substantially insoluble or only sparsely soluble in water. The poor water-solubility of cannabinoids results in major difficulties in formulation and presents a major challenge to consistent drug delivery.

Furthermore, when administered orally in the form of an oil solution or some kind of water and/or oil suspension or emulsion, lipophilic compounds usually show poor bioavailability. The bioavailability of a drug depends on several parameters, i.e., the physicochemical nature of the active compound, the dosage form, as well as physiological factors. The cannabinoid compounds, being hydrophobic in nature, show wetting difficulties and poor dissolution in the gastrointestinal region.

Oral compositions comprising synthetic THC, e.g. gelatine capsules and tablets, are also known in the art. Marinol® (active component is dronabinol) is a soft gelatine capsule comprising synthetic THC. The bioavailability of Marinol® after oral intake is only approximately 15%. Namisol® (active component is dronabinol) is a THC sublingual tablet (ultra pure extract from Cannabis sativa), which is claimed to have a rapid uptake through the sublingual mucosa. The problem is that the tablet has to be kept under the tongue for the time it takes to dissolve in the saliva. Cesamet® (active component is nabilone) is a capsule comprising the synthetic cannabinoid nabilone. It is said to have fewer undesired side effects than THC.

Sativex® comprises THC and CBD and is commercially available as a buccal mouth spray for multiple sclerosis and for the alleviation of pain. Each spray of Sativex® delivers a fixed dose of 2.7 mg THC and 2.5 mg CBD. It is reported to cause irritation of the oral mucosa (20-25% of the patients) and to have a bad taste because of the high ethanol content.

Accordingly, there is a need for developing oral formulations of cannabinoids with improved delivery and enhanced bioavailability by masking the unpleasant taste of these lipophilic cannabinoids.

Therefore, object of the present invention is to provide more optimized and improved delivery systems for cannabinoids to meet the desired needs of the patients. It is still another object of the present invention to provide oral dosage forms which provides sufficient delivery of cannabinoids for better efficacy and sustained delivery of cannabinoids for prolonged efficacy in the treatment of neurological diseases and disorders.

### SUMMARY

In one aspect, there is provided a Self Emulsifying Drug Delivery System, commonly known as SEDDS, suitable for oral administration. More particularly, the present invention is directed to pharmaceutical cannabinoid compositions suitable for oral administration, in the form of emulsion pre-concentrates, comprising:
a. a cannabinoid at 0.01-10% (w/w),
b. a solubilizing agent at 20-40% (w/w), and
c. a stabilizer at 0.01-5% (w/w)
   wherein the said oral cannabinoid compositions forms an in-situ emulsion upon contact with aqueous media such as gastrointestinal fluids.

In an embodiment of the oral cannabinoid compositions as described herein, the cannabinoid is cannabidiol (CBD), cannabidiolic acid (CBDA), cannabidivarin (CBDV), cannabigerol (CBG), cannabigervarin (CBGV), tetrahydrocannabivarin (THCV), tetrahydrocannabinol (THC), cannabinol (CBN), or any combination thereof.

In an embodiment of the oral cannabinoid compositions as described herein, the cannabinoid is cannabidiol (CBD) alone or in combination with tetrahydrocannabinol (THC).

In an embodiment of the oral cannabinoid compositions as described herein, the solubilizing agent is polyoxyl castor oil.

In an embodiment of the oral cannabinoid compositions as described herein, the stabilizer is citric acid used to maintain the pH and to prevent the oxidation of the cannabinoids.

In an embodiment of the oral cannabinoid compositions as described herein, the compositions are emulsion pre-concentrates at the time of administration to a subject. The emulsion pre-concentrates are filled into single unit dosage forms such as capsules, drinking ampoules and dose cushions, or may alternatively be formed as other suitable dosage forms such as powder, granules, pellets, tablets, chewable soft pills, and chewy-base lozenges.

In an embodiment of the oral cannabinoid compositions as described herein, the compositions are formed as liquid, solid and powder compositions.

In an embodiment of the oral cannabinoid compositions as described herein, the liquid compositions further comprise one or more of:
a. an oil or semi-solid fat at 1-20% (w/w), and
b. a solvent to make 100% by weight.

In an embodiment of the liquid cannabinoid compositions as described herein, the oil or semi-solid fat is a medium chain triglyceride oil (MCT oil) extracted from coconut oil and solvent is propylene glycol.

In an embodiment of the liquid cannabinoid compositions as described herein, the liquid compositions are soluble in water.

In an embodiment of the oral cannabinoid compositions as described herein, the solid compositions further comprise one or more of:
a. a surfactant at 1-15% (w/w),
b. an oil or semi-solid fat at 1-20% (w/w),
c. a solvent at 1-5% (w/w), and
d. a co-solvent to make 100% by weight.

In an embodiment of the solid cannabinoid compositions as described herein, the surfactant is Poloxamer, oil or semi-solid fat is a medium chain triglyceride oil (MCT oil) extracted from coconut oil, solvent is propylene glycol and co-solvent is polyethylene glycol.

In an embodiment of the oral cannabinoid compositions as described herein, the powder compositions further comprise one or more of:
a. a surfactant at 20-40% (w/w),
b. a solvent at 1-5% (w/w), and
c. a thickening agent at 20-30% (w/w).

In an embodiment of the powder cannabinoid compositions as described herein, the surfactant is Poloxamer, solvent is propylene glycol and thickening agent is silicon dioxide.

In an embodiment of the oral cannabinoid compositions as described herein, the powder compositions are also formulated into controlled release tablets by melt granulation process using a standardized matrix to control the release of cannabinoids upon contact with aqueous media such as gastrointestinal fluids.

In an embodiment of the oral cannabinoid compositions as described herein, the controlled release tablet compositions further comprise one or more of:
a. a carrier at 20-40% (w/w),
b. a gelling agent at 1-10% (w/w),
c. a flavoring agent at 1-10% (w/w),
d. a plasticizer at 1-10% (w/w),
e. a lubricant at 1-2% (w/w), and
f. a filler to make 100% by weight.

In an embodiment of the controlled release tablet compositions as described herein, the carrier is silicon dioxide, the gelling agent is Carbopol® 974 polymer, the flavoring agent is spearmint, the plasticizer is polyvinylpyrrolidone (PVP), the lubricant is magnesium stearate, and the filler is microcrystalline cellulose.

In another aspect, there is provided use of the oral cannabinoid formulations described herein for the treatment of neurological diseases and disorders in a subject.

In an embodiment of the use as described herein, the neurological diseases and disorders include but are not limited to headaches; depression; anxiety; epileptic seizures; movement disorders; dementias; sleep disorders; Amyotrophic Lateral Sclerosis (ALS); stroke; Parkinson's Disease; neuropathic pain due to amputation; cancer; carpal tunnel syndrome; chemotherapy; diabetes; facial nerve problems; HIV infection/AIDS; multiple myeloma; multiple sclerosis; nerve or spinal cord compression from herniated discs or from arthritis in the spine; shingles; spine surgery; syphilis; thyroid problems or vitamin B deficiency.

Other aspects, features, and embodiments of the present disclosure will become apparent to those of ordinary skill in the art upon review of the following description of specific embodiments in conjunction with the accompanying figures.

### DETAILED DESCRIPTION

Provided herein pharmaceutical cannabinoid compositions suitable for oral administration, in the form of emulsion pre-concentrates which forms an in-situ emulsion upon contact with aqueous media such as gastrointestinal fluids.

Compositions provided herein comprise the combination of a surfactants, solubilizers and carriers, which are selected such that these ingredients not only improve the solubility of cannabinoids in the formulation, but they also enhance the degree and rate of cannabinoids absorption as well as control the release of cannabinoids in the systemic circulation.

Also provided herein is a Self-emulsifying drug delivery system (SEDDS) to enhance the bioavailability of lipophilic cannabinoid compounds to achieve better therapeutic efficacy.

Compositions provided herein may be useful for treating neurological disease and/or disorder in a subject.

Compositions provided herein can also be used in conjunction with available treatments of neurological diseases and/or disorders.

Oral cannabinoid compositions provided herein exhibit excellent overall stability, dissolution, and bioavailability profile.

“Cannabinoid” as used herein, is meant to include compounds which interact with the cannabinoid receptor and various cannabinoid mimetics, such as cannabidiol (CBD), cannabidiolic acid (CBDA), cannabidivarin (CBDV), cannabigerol (CBG), cannabigervarin (CBGV), tetrahydrocannabivarin (THCV), tetrahydrocannabinol (THC) and cannabinol (CBN).

“Phytocannabinoids” as used herein means cannabinoids extracted from Cannabis plant species including by the way of non-limiting example Cannabis sativa, Cannabis indica and Cannabis ruderalis and all resins, stalks, flowers, seeds, and oils related thereto.

The term “active agent” is generally understood to mean an active pharmaceutical ingredient.

As used herein, the terms “active ingredient,” “active pharmaceutical ingredient,” or “active pharmaceutical agent” refer to an agent, active ingredient, compound, or substance, compositions, or mixtures thereof, that provide a pharmacological, often beneficial, effect. Reference to a specific active ingredient includes, where appropriate, the active ingredient and any of its pharmaceutically acceptable free acids, free bases, salts, or esters.

The term “formulation” or “composition” as used herein refers to the active pharmaceutical ingredient, nutraceutical, nutritional, vitamin, or drug in combination with pharmaceutically acceptable excipients. This includes orally administrable formulations as well as formulations administrable by other means.

The term “excipient” herein means any substance, not itself an active agent, which may be used as a carrier or vehicle for delivery of an active agent to a subject or combined with an active agent to improve its handling or storage properties or to permit or facilitate formation of a dose unit of the compositions. Examples of excipients include, but are not limited to, a “thickening agent”, which is capable of increasing the viscosity of a composition; a “stabilizer”, which prevents decomposition of composition by microbial growth or by undesirable chemical changes; a “surfactant”, which act as solubilizing agent by lowering the surface tension (or interfacial tension) between two liquids or between a liquid and a solid; a “solubilizing agent” refers to inactive pharmaceutical ingredients used to improve solubility of poorly water-soluble drugs such as cannabinoids; a “carrier”, which is primarily used to control the release of a drug into systemic circulation; a “gel-forming agent”, which is capable of forming a semi-crystalline structure by reaction with another material or by lowering of the temperature thereof while dissolved or colloidally suspended in a liquid medium; a “flavoring agent” which is a single chemical entity or a blend of chemicals of synthetic or natural origin that can produce a taste or aroma (i.e. fragrance) when consumed orally or inhaled; a “plasticizer” that can be used to coat a solid dosage formulation; a “lubricant” that can be used to enhance the powder flow by reducing the inter-particle friction in a solid dosage formulation such as tablets; a “filler” to make a product bigger or easier to handle, for example, fillers are often used to make pills or capsules because the amount of active drug is too small to be handled conveniently.

As used herein, the terms “dosage” or “dose” denote any form of the active ingredient formulation that contains an amount sufficient to produce a therapeutic effect with a single administration. The dosage form used herein is for oral administration. The preferred oral dosage forms are soft capsules or enteric soft capsules and tablets.

The term “drug delivery system” refers to a formulation or device that delivers therapeutic agent(s) to desired body location(s) and/or provides timely release of therapeutic agent(s).

The term “self-emulsifying” or “self-emulsifying drug delivery system (SEDDS)” refers to isotropic mixtures of natural or synthetic oils, solid or liquid surfactants, or alternatively, one or more hydrophilic solvents and co-solvents/surfactants. The combination of a pharmaceutical oil and a surfactant can provide a formulation, which emulsifies and disperses rapidly in the gastrointestinal fluid. Upon mild agitation followed by dilution in aqueous media, these systems can form fine oil-in-water (o/w) emulsions with a very small droplet size.

The term “therapeutically effective amount” or “therapeutically and/or prophylactically effective amount” as used herein refers to an amount of compound or agent that is sufficient to elicit the required or desired therapeutic and/or prophylactic response, as the particular treatment context may require. It will be understood that a therapeutically and/or prophylactically effective amount of an active agent for a subject is dependent inter alia on the body weight of the subject as well as other factors known to a person of ordinary skill in the art.

The terms “treat,” “treating,” or “treatment of” are used herein in their broad senses unless otherwise specifically indicated in the particular context, and results of a treatment may generally include reversing, alleviating, or inhibiting the progress of an indicated disorder or condition, or one or more symptoms of the disorder or condition.

“wt %” or “w/w %” when referring to the percentage of a component in a composition is percentage of the weight of the component in the composition relative to the total weight of the composition.

“Alleviate” as used herein, is meant to include complete elimination as well as any clinically or quantitatively measurable reduction in the subject's symptoms and/or discomfort.

A “subject” herein to which a therapeutic agent or compositions thereof can be administered includes mammals such as a human subject of either sex and of any age, and also includes any nonhuman animal, particularly a domestic or companion animal such as a cat, dog or horse, as well as laboratory animals such as rats or guinea pigs.

As used herein, the term “bioavailability” refers to the proportion of an active pharmaceutical ingredient that enters the systemic circulation when introduced into the body and is able to have a physiological effect.

As used herein, the term “enhanced bioavailability” refers to the increased proportion of an active pharmaceutical ingredient that enters the systemic circulation when introduced into the body as compared to a reference's bioavailability.

The term “stable compositions(s)” as used herein, refers to a composition that does not show any precipitation in Fasted-State Simulated Gastric Fluid (FaSSGF) at pH 2.0, temperature of 37° C.±0.5° C. and under stirring at a speed of 50 rpm at least for 60 minutes. Also, the term “stable compositions(s)” refers to a composition which upon subjected to stability evaluation at 40° C. and 75% RH (relative humidity) or 25° C. and 60% RH (relative humidity), is substantially free of impurities, or comprises not more than 5% impurities, or comprises impurities levels which are acceptable by regulatory bodies such as US FDA.

As used herein, the term “dissolution rate” or “dispersion rate” refers to the amount of time it takes for an active ingredient (e.g., cannabidiol) or compositions thereof to dissolve in a solvent. The dissolution rate may depend on a variety of factors including mixing, temperature, pH, solvent, particle size, etc. The dissolution rate of a drug or compositions thereof affects the bioavailability of the drug. In certain circumstances, dissolution rate is used to determine drug availability from solid dosage forms.

As used herein, the terms “droplet size” and “particle size” both refer to the diameter of the respective droplet or particle, unless otherwise specifically noted.

As used herein, the terms “sustained release”, “controlled release”, “prolonged release”, “delayed release”, “retarded release”, or “timed release” are used to indicate that control is exercised over both the duration and profile of the in vivo drug release curve. It is intended to mean that the composition require at least an hour to release a major portion of the active ingredient into the surrounding medium, e.g., about 1-6 hours.

The terms “neurological disease”, or “neurological disorder,” as used herein, refers to a heterogeneous group of neurological conditions that result from damage to the nervous system.

The term “efficacy” refers to the effectiveness of a particular active agent for its intended purpose, i.e., the ability of a given active agent to cause its desired pharmacologic effect.

The term “safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

The terms “tolerable” and “tolerability” refer to the ability of the pharmaceutical compositions of the present invention to not elicit an adverse reaction in the subject to whom such compositions is administered, or alternatively not to elicit a serious adverse reaction in the subject to whom such compositions is administered.

As discussed in greater detail in the illustrative and non-limiting examples provided herein, the present disclosure is directed to topical formulations/compositions that incorporate at least one cannabinoid.

In one aspect, there are provided oral cannabinoid compositions comprising:
a. a cannabinoid at 0.01-10% (w/w),
b. a solubilizing agent at 20-40% (w/w), and
c. a stabilizer at 0.01-5% (w/w)
   wherein the said oral cannabinoid compositions forms an in-situ emulsion upon contact with aqueous media such as gastrointestinal fluids.

Exemplary cannabinoids include cannabidiol (CBD), cannabidiolic acid (CBDA), cannabidivarin (CBDV), cannabigerol (CBG), cannabigervarin (CBGV), tetrahydrocannabivarin (THCV), tetrahydrocannabinol (THC), cannabinol (CBN), combinations, and mixtures thereof extracted from Cannabis plant species including Cannabis sativa, Cannabis indica and Cannabis ruderalis and all resins, stalks, flowers, seeds, and oils related thereto.

In certain exemplary, non-limiting embodiments, oral cannabinoid compositions provided herein may include about 0.01% to about 10% (w/w) of cannabinoid(s). For example, oral cannabinoid compositions provided herein may comprise about 0.01% to about 10%, 0.1% to about 10%, about 0.5% to about 10%, about 1% to about 10%, about 2% to about 10%, about 3% to about 10%, about 4% to about 10%, about 5% to about 10%, about 6% to about 10%, about 7% to about 10%, about 8% to about 10%, about 9% to about 10%, about 0.01% to about 9%, about 0.1% to about 9%, about 0.5% to about 9%, about 1% to about 9%, about 2% to about 9%, about 3% to about 9%, about 4% to about 9%, about 5% to about 9%, about 6% to about 9%, about 7% to about 9%, about 8% to about 9%, about 0.01% to about 8%, about 0.1% to about 8%, about 0.5% to about 8%, about 1% to about 8%, about 2% to about 8%, about 3% to about 8%, about 4% to about 8%, about 5% to about 8%, about 6% to about 8%, about 7% to about 8%, about 0.01% to about 7%, about 0.1% to about 7%, about 0.5% to about 7%, about 1% to about 7%, about 2% to about 7%, about 3% to about 7%, about 4% to about 7%, about 5% to about 7%, about 6% to about 7%, about 0.01% to about 6%, about 0.1% to about 6%, about 0.5% to about 6%, about 1% to about 6%, about 2% to about 6%, about 3% to about 6%, about 4% to about 6%, about 5% to about 6%, about 0.01% to about 5%, about 0.1% to about 5%, about 0.5% to about 5%, about 1% to about 5%, about 2% to about 5%, about 3% to about 5%, about 4% to about 5%, about 0.01% to about 4%, about 0.1% to about 4%, about 0.5% to about 4%, about 1% to about 4%, about 2% to about 4%, about 3% to about 4%, about 0.01% to about 3%, about 0.1% to about 3%, about 0.5% to about 3%, about 1% to about 3%, about 2% to about 3%, about 0.01% to about 2%, about 0.1% to about 2%, about 0.5% to about 2%, about 1% to about 2%, about 0.01% to about 1%, about 0.1% to about 1%, about 0.5% to about 1%, about 0.01% to about 0.5%, about 0.1% to about 0.5% or about 0.01% to about 0.1% (w/w) of cannabinoid(s).

In certain exemplary, non-limiting embodiments, oral cannabinoid compositions provided herein may comprise at least 0.01%, at least 0.1%, at least 0.5%, at least 1%, at least 2%, at least 3%, at least 4%, at least 5%, at least 6%, at least 7%, at least 8%, at least 9% or at least 10% (w/w) of cannabinoid(s).

In certain exemplary, non-limiting embodiments, oral cannabinoid compositions provided herein may comprise about 0.01%, about 0.1%, about 0.5%, about 1%, about 2%, about 3%, about 4%, about 5%, about 6%, about 7%, about 8%, about 9% or about 10% (w/w) of cannabinoid(s).

Without being limited by any particular theory, it is expected that cannabinoid compositions provided herein enhance the proportion of cannabinoids in systemic circulation when introduced into the body through self-emulsification and are able to produce a physiological effect.

Other ingredients may be provided in oral cannabinoid compositions provided herein, so long as they are physiologically acceptable and suitable for use in combination with cannabinoids.

In certain exemplary, non-limiting embodiments, oral cannabinoid compositions provided herein may include one or more solubilizing agent(s), such as polyoxyl castor oil, Poloxamer, polysorbates, benzalkonium chloride, cyclodextrins, lecithin, benzyl alcohol, benzyl benzoate, combinations, and mixtures thereof.

In certain exemplary, non-limiting embodiments, oral cannabinoid compositions provided herein may include about 20% to about 30% (w/w) of solubilizing agent(s). For example, oral cannabinoid compositions provided herein may comprise about 20% to about 30%, about 21% to about 30%, about 22% to about 30%, about 23% to about 30%, about 24% to about 30%, about 25% to about 30%, about 26% to about 30%, about 27% to about 30%, about 28% to about 30%, about 29% to about 30%, about 20% to about 29%, about 21% to about 29%, about 22% to about 29%, about 23% to about 29%, about 24% to about 29%, about 25% to about 29%, about 26% to about 29%, about 27% to about 29%, about 28% to about 29%, about 20% to about 28%, about 21% to about 28%, about 22% to about 28%, about 23% to about 28%, about 24% to about 28%, about 25% to about 28%, about 26% to about 28%, about 27% to about 28%, about 20% to about 27%, about 21% to about 27%, about 22% to about 27%, about 23% to about 27%, about 24% to about 27%, about 25% to about 27%, about 26% to about 27%, about 20% to about 26%, about 21% to about 26%, about 22% to about 26%, about 23% to about 26%, about 24% to about 26%, about 25% to about 26%, about 20% to about 25%, about 21% to about 25%, about 22% to about 25%, about 23% to about 25%, about 24% to about 25%, about 20% to about 24%, about 21% to about 24%, about 22% to about 24%, about 23% to about 24%, about 20% to about 23%, about 21% to about 23%, about 22% to about 23%, about 20% to about 22%, about 21% to about 22% or about 20% to about 21% (w/w) of solubilizing agent(s).

In certain exemplary, non-limiting embodiments, oral cannabinoid compositions provided herein may comprise at least 20%, at least 21%, at least 22%, at least 23%, at least 24%, at least 25%, at least 26%, at least 27%, at least 28%, at least 29% or at least 30% (w/w) of solubilizing agent(s).

In certain exemplary, non-limiting embodiments, oral cannabinoid compositions provided herein may comprise about 20%, about 21%, about 22%, about 23%, about 24%, about 25%, about 26%, about 27%, about 28%, about 29% or about 30% (w/w) of solubilizing agent(s).

In certain exemplary, non-limiting embodiments, oral cannabinoid compositions provided herein may include one or more stabilizer(s) to maintain the pH and to prevent the oxidation of the cannabinoids, which may comprise organic acids, carboxylic acids, acid salts of amino acids such as cysteine hydrochloride, glycine hydrochloride, cystine dihydrochloride, ascorbic acid, malic acid, isoascorbic acid, citric acid, tartaric acid, palmityl, stearyl ascorbate, combinations, and mixtures thereof.

In certain exemplary, non-limiting embodiments, oral cannabinoid compositions provided herein may include about 0.01% to about 5% (w/w) of stabilizer(s). For example, oral cannabinoid compositions provided herein may comprise about 0.01% to about 5%, about 0.1% to about 5%, about 0.5% to about 5%, about 1% to about 5%, about 2% to about 5%, about 3% to about 5%, about 4% to about 5%, about 0.01% to about 4%, about 0.1% to about 4%, about 0.5% to about 4%, about 1% to about 4%, about 2% to about 4%, about 3% to about 4%, about 0.01% to about 3%, about 0.1% to about 3%, about 0.5% to about 3%, about 1% to about 3%, about 2% to about 3%, about 0.01% to about 2%, about 0.1% to about 2%, about 0.5% to about 2%, about 1% to about 2%, about 0.01% to about 1%, about 0.1% to about 1%, about 0.5% to about 1%, about 0.01% to about 0.5%, about 0.1% to about 0.5%, or about 0.01% to about 0.1% (w/w) of stabilizer(s).

In certain exemplary, non-limiting embodiments, oral cannabinoid compositions provided herein may comprise at least 0.01%, at least 0.1%, at least 0.5%, at least 1%, at least 2%, at least 3%, at least 4%, or at least 5% (w/w) of stabilizer(s).

In certain exemplary, non-limiting embodiments, oral cannabinoid compositions provided herein may comprise about 0.01%, about 0.1%, about 0.5%, about 1%, about 2%, about 3%, about 4% or about 5% (w/w) of stabilizer(s).

In certain exemplary, non-limiting embodiments, oral cannabinoid compositions provided herein may form emulsion pre-concentrates at the time of administration to a subject. The emulsion pre-concentrates may be filled into single unit dosage forms such as capsules, drinking ampoules and dose cushions, or may alternatively be formed as other suitable dosage forms such as powder, granules, pellets, tablets, chewable soft pills, and chewy-base lozenges.

In certain exemplary, non-limiting embodiments, cannabinoid compositions provided herein may be provided as oral self-emulsifying capsules, liquid, powder, or tablets.

In certain exemplary, non-limiting embodiments, cannabinoid compositions provided herein may be formulated as liquid, solid or powdered forms.

In certain exemplary, non-limiting embodiments, the liquid oral cannabinoid compositions comprise oil(s) or semi-solid fat(s), which may comprise a variety of different oils e.g., soybean oil, olive oil, fish oil, fish oil extract, safflower oil, corn oil, sunflower oil, coconut oil, palm kernel oil, rapeseed oil, sesame oil, vegetable oils, mineral oils, medium chain triglycerides (MCT), combinations and mixtures thereof.

In certain exemplary, non-limiting embodiments, the liquid oral cannabinoid compositions provided herein may include about 1% to about 20% (w/w) of oil(s) or semi-solid fat(s).

In certain exemplary, non-limiting embodiments, the liquid oral cannabinoid compositions provided herein may comprise at least 1%, at least 2%, at least 3%, at least 4%, at least 5%, at least 6%, at least 7%, at least 8% (w/w), at least 9%, at least 10%, at least 11%, at least 12%, at least 13%, at least 14%, at least 15%, at least 16%, at least 17%, at least 18%, at least 19%, or at least 20% of oil(s) or semi-solid fat(s).

In certain exemplary, non-limiting embodiments, the liquid oral cannabinoid compositions provided herein may comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% (w/w) of oil(s) or semi-solid fat(s).

In certain exemplary, non-limiting embodiments, the liquid oral cannabinoid compositions provided herein comprise pharmaceutically acceptable solvent(s) which may be selected from the group consisting of diethylene glycol ethyl ether, methoxy polyethylene glycol, propylene glycol, polypropylene glycol, polyethylene glycol (PEG), glycerol, ethanol, dimethyl isosorbide, glycofurol, propylene carbonate, dimethyl acetamide, combinations or mixtures thereof.

In another aspect, the liquid cannabinoid compositions comprise:
a. a cannabinoid at 0.01-10% (w/w),
b. a solubilizing agent at 20-40% (w/w),
c. a stabilizer at 0.01-5% (w/w),
d. an oil or semi-solid fat at 1-20% (w/w), and
e. a solvent to make 100% by weight.

In certain exemplary, non-limiting embodiments, the liquid oral cannabinoid compositions are soluble in water.

In certain exemplary, non-limiting embodiments, the solid oral cannabinoid compositions comprise herein may include one or more surfactant(s), which may be a non-ionic surfactant. Examples of non-ionic surfactants include polysorbate 20 (Tween 20), polysorbate 40 (Tween 40), polysorbate 60 (Tween 60), polysorbate 80 (Tween 80), Poloxamer, combinations, and mixtures thereof.

In certain exemplary, non-limiting embodiments, the solid oral cannabinoid compositions provided herein may include about 1% to about 15% (w/w) of surfactant(s).

In certain exemplary, non-limiting embodiments, the solid oral cannabinoid compositions provided herein may comprise at least 1%, at least 2%, at least 3%, at least 4%, at least 5%, at least 6%, at least 7%, at least 8% (w/w), at least 9%, at least 10%, at least 11%, at least 12%, at least 13%, at least 14%, or at least 15% of surfactant(s).

In certain exemplary, non-limiting embodiments, the solid oral cannabinoid compositions provided herein may comprise about 1%, about 2%, about 3%, about 4%, about 5%, about 6%, about 7%, about 8%, about 9%, about 10%, about 11%, about 12%, about 13%, about 14%, or about 15% of surfactant(s).

In certain exemplary, non-limiting embodiments, the solid oral cannabinoid compositions comprise herein may include oil(s) or semi-solid fat(s), which may comprise a variety of different oils e.g., soybean oil, olive oil, fish oil, fish oil extract, safflower oil, corn oil, sunflower oil, coconut oil, palm kernel oil, rapeseed oil, sesame oil, vegetable oils, mineral oils, medium chain triglycerides (MCT), combinations and mixtures thereof.

In certain exemplary, non-limiting embodiments, the solid oral cannabinoid compositions provided herein may include about 1% to about 20% (w/w) of oil(s) or semi-solid fat(s).

In certain exemplary, non-limiting embodiments, the solid oral cannabinoid compositions provided herein may comprise at least 1%, at least 2%, at least 3%, at least 4%, at least 5%, at least 6%, at least 7%, at least 8% (w/w), at least 9%, at least 10%, at least 11%, at least 12%, at least 13%, at least 14%, at least 15%, at least 16%, at least 17%, at least 18%, at least 19%, or at least 20% of oil(s) or semi-solid fat(s).

In certain exemplary, non-limiting embodiments, the solid oral cannabinoid compositions provided herein may comprise about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% (w/w) of oil(s) or semi-solid fat(s).

In certain exemplary, non-limiting embodiments, the solid oral cannabinoid compositions provided herein may comprise pharmaceutically acceptable solvent(s) and co-solvent(s) which may be selected from the group consisting of diethylene glycol ethyl ether, methoxy polyethylene glycol, propylene glycol, polypropylene glycol, polyethylene glycol (PEG), glycerol, ethanol, dimethyl isosorbide, glycofurol, propylene carbonate, dimethyl acetamide, combinations or mixtures thereof.

In another aspect, the solid cannabinoid compositions comprise:
a. a cannabinoid at 0.01-10% (w/w),
b. a solubilizing agent at 20-40% (w/w),
c. a stabilizer at 0.01-5% (w/w),
d. a surfactant at 1-15% (w/w),
e. an oil or semi-solid fat at 1-20% (w/w),
f. a solvent at 1-5% (w/w), and
g. a co-solvent to make 100% by weight.

In certain exemplary, non-limiting embodiments, the powdered oral cannabinoid compositions comprise herein may include one or more surfactant(s), which may be a non-ionic surfactant. Examples of non-ionic surfactants include polysorbate 20 (Tween 20), polysorbate 40 (Tween 40), polysorbate 60 (Tween 60), polysorbate 80 (Tween 80), Poloxamer, combinations, and mixtures thereof.

In certain exemplary, non-limiting embodiments, the powdered oral cannabinoid compositions provided herein may include about 20% to about 40% (w/w) of surfactant(s).

In certain exemplary, non-limiting embodiments, the powdered oral cannabinoid compositions provided herein may comprise at least 20%, at least 21%, at least 22%, at least 23%, at least 24%, at least 25%, at least 26%, at least 27% (w/w), at least 28%, at least 29%, at least 30%, at least 31%, at least 32%, at least 33%, at least 34%, at least 35%, at least 36%, at least 37%, at least 38%, at least 39%, or at least 40% of surfactant(s).

In certain exemplary, non-limiting embodiments, the powdered oral cannabinoid compositions provided herein may comprise about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% of surfactant(s).

In certain exemplary, non-limiting embodiments, the powdered oral cannabinoid compositions provided herein may comprise one or more pharmaceutically acceptable solvent(s) which may be selected from the group consisting of diethylene glycol ethyl ether, methoxy polyethylene glycol, propylene glycol, polypropylene glycol, polyethylene glycol (PEG), glycerol, ethanol, dimethyl isosorbide, glycofurol, propylene carbonate, dimethyl acetamide, combinations or mixtures thereof.

In certain exemplary, non-limiting embodiments, the powdered oral cannabinoid compositions provided herein may comprise about 1% to about 5% (w/w) of solvent(s).

In certain exemplary, non-limiting embodiments, the powdered oral cannabinoid compositions provided herein may comprise at least 1%, at least 2%, at least 3%, at least 4%, or at least 5% (w/w) of solvent(s).

In certain exemplary, non-limiting embodiments, the powdered oral cannabinoid compositions provided herein may comprise about 1%, about 2%, about 3%, about 4%, or about 5% (w/w) of solvent(s).

In certain exemplary, non-limiting embodiments, the powdered oral cannabinoid compositions provided herein may comprise one or more thickening agent(s) which may be selected from the group consisting of bentones, gums, silicon dioxide, stearyl alcohol, castor wax, combinations, or mixtures thereof.

In certain exemplary, non-limiting embodiments, the powdered oral cannabinoid compositions provided herein may comprise about 20% to about 30% (w/w) of thickening agent(s).

In certain exemplary, non-limiting embodiments, the powdered oral cannabinoid compositions provided herein may comprise at least 20%, at least 21%, at least 22%, at least 23%, at least 24%, at least 25%, at least 26%, at least 27% (w/w), at least 28%, at least 29%, or at least 30% (w/w) of thickening agent(s).

In certain exemplary, non-limiting embodiments, the powdered oral cannabinoid compositions provided herein may comprise about 20%, about 21%, about 22%, about 23%, about 24%, about 25%, about 26%, about 27%, about 28%, about 29%, or about 30% (w/w) of thickening agent(s).

In another aspect, the powdered cannabinoid compositions comprise:
a. a cannabinoid at 0.01-10% (w/w),
b. a solubilizing agent at 20-40% (w/w),
c. a stabilizer at 0.01-5% (w/w),
d. a surfactant at 20-40% (w/w),
e. a solvent at 1-5% (w/w), and
f. a thickening agent at 20-30% (w/w).

In certain exemplary, non-limiting embodiments, the powdered oral cannabinoid compositions provided herein may also be formulated into controlled release tablets by melt granulation process using a standardized matrix to control the release of cannabinoids upon contact with aqueous media such as gastrointestinal fluids.

In certain exemplary, non-limiting embodiments, the controlled release tablet cannabinoid compositions further comprise one or more of:
a. a carrier at 20-40% (w/w),
b. a gelling agent at 1-10% (w/w),
c. a flavoring agent at 1-10% (w/w),
d. a plasticizer at 1-10% (w/w),
e. a lubricant at 1-2% (w/w), and
f. a filler to make 100% by weight.

In certain exemplary, non-limiting embodiments, the controlled release tablet cannabinoid compositions comprise one or more carrier(s), which may be selected from the group consisting of bentones, gums, silicon dioxide, stearyl alcohol, castor wax, combinations, or mixtures thereof.

In certain exemplary, non-limiting embodiments, the controlled release tablet cannabinoid compositions comprise one or more gelling agent(s) selected from the group consisting of the family of polyacrylamides; copolymers of acrylic acid; “electrolyte-insensitive” carbomers; polysaccharides; cellulose and derivatives thereof; and magnesium aluminum silicates.

Examples include sodium acryloyldimethyltaurate copolymer/isohexadecane/polysorbate 80 mixture, the polyacrylamide/C13-14 isoparaffin/laureth-7 mixture, Carbopol 1382, Carbopol 974, Carbopol 980, Pemulen™ TR, xanthan gum, hydroxypropylmethylcellulose or hydroxyethylcellulose, combinations, and mixtures thereof.

In certain exemplary, non-limiting embodiments, the controlled release tablet cannabinoid compositions comprise one or more flavoring agent(s) selected from group consisting of agents such as caraway, clove, lemon, spearmint, rose, peppermint, ginger, raspberry, maltol, syrups, cherry, combinations, or mixtures thereof.

In certain exemplary, non-limiting embodiments, the controlled release tablet cannabinoid compositions comprise one or more plasticizer(s) which may be a polymer. Examples of polymers include hydroxypropyl-methylcellulose (HPMC), polyvinylpyrrolidone (PVP), combinations or mixtures thereof.

In certain exemplary, non-limiting embodiments, the controlled release tablet cannabinoid compositions comprises one or more lubricant(s) which may be metallic salts of fatty acids such as magnesium stearate, calcium stearate, and zinc stearate; or fatty acids such as stearic acid; or fatty acid esters, such as glyceride esters (glyceryl monostearate, glyceryl tribehenate, and glyceryl dibehenate) and sugar esters (sorbitan monostearate and sucrose monopalmitate), combinations or mixtures thereof.

In certain exemplary, non-limiting embodiments, the controlled release tablet cannabinoid compositions comprise one or more filler(s) selected from group consisting of gelatin, microcrystalline cellulose powder, carrageenan, titanium dioxide, combinations, or mixtures thereof.

It is understood that the amount of cannabinoid necessary to achieve a desired therapeutic result is influenced by, and will therefore vary based on, a number of factors, including for example and without limitation, the age, sex, and weight of the subject, factors that influence the metabolic rate, and the specific conditions, diseases, or related treatment symptoms of the subject. The concentration of at least one cannabinoid in compositions provided herein is between about 0.01% and about 10% (w/w).

One of skill in the art will understand that the ingredients in the final formulations must total 100% and, based on the teachings provided herein, will understand that modifications to the exemplary formulations provided herein are possible (e.g., replacement of a recited ingredient with a different ingredient, addition of a different ingredient, and/or modification of an amount of an ingredient) provided that such modifications result in a formulation as taught and described herein (i.e. capable of delivering an active agent such a cannabinoid to the site of action).

In certain exemplary, non-limiting embodiments, oral cannabinoid compositions may include cannabinoids in a specific therapeutic amount for treating neurological disease and/or disorder in a subject.

In certain exemplary, non-limiting embodiments, the neurological diseases and disorders include but are not limited to headaches; depression; anxiety; epileptic seizures; movement disorders; dementias; sleep disorders; Amyotrophic Lateral Sclerosis (ALS); stroke; Parkinson's Disease; neuropathic pain due to amputation; cancer; carpal tunnel syndrome; chemotherapy; diabetes; facial nerve problems; HIV infection/AIDS; multiple myeloma; multiple sclerosis; nerve or spinal cord compression from herniated discs or from arthritis in the spine; shingles; spine surgery; syphilis; thyroid problems or vitamin B deficiency.

The discussion herein and the following Examples set forth and illustrate various exemplary embodiments of the present disclosure, which are understood to be illustrative and non-limiting.

Non-limiting Embodiments

Particular embodiments of the disclosure include, without limitation, the following:

1. Oral cannabinoid compositions comprising:
a. a cannabinoid at 0.01-10% (w/w),
b. a solubilizing agent at 20-40% (w/w), and
c. a stabilizer at 0.01-5% (w/w)

2. The oral cannabinoid compositions of embodiment 1, which comprises about 0.01% (w/w) of the cannabinoid.

3. The oral cannabinoid compositions of embodiment 1, which comprises about 0.1% (w/w) of the cannabinoid.

4. The oral cannabinoid compositions of embodiment 1, which comprises about 0.5% (w/w) of the cannabinoid.

5. The oral cannabinoid compositions of embodiment 1, which comprises about 1% (w/w) of the cannabinoid.

6. The oral cannabinoid compositions of embodiment 1, which comprises about 2% (w/w) of the cannabinoid.

7. The oral cannabinoid compositions of embodiment 1, which comprises about 3% (w/w) of the cannabinoid.

8. The oral cannabinoid compositions of embodiment 1, which comprises about 4% (w/w) of the cannabinoid.

9. The oral cannabinoid compositions of embodiment 1, which comprises about 5% (w/w) of the cannabinoid.

10. The oral cannabinoid compositions of embodiment 1, which comprises about 6% (w/w) of the cannabinoid.

11. The oral cannabinoid compositions of embodiment 1, which comprises about 7% (w/w) of the cannabinoid.

12. The oral cannabinoid compositions of embodiment 1, which comprises about 8% (w/w) of the cannabinoid.

13. The oral cannabinoid compositions of embodiment 1, which comprises about 9% (w/w) of the cannabinoid.

14. The oral cannabinoid compositions of embodiment 1, which comprises about 10% (w/w) of the cannabinoid.

15. The oral cannabinoid compositions of any one of the embodiments 1 to 14, wherein the cannabinoid is cannabidiol (CBD), cannabidiolic acid (CBDA), cannabidivarin (CBDV), cannabigerol (CBG), cannabigervarin (CBGV), tetrahydrocannabivarin (THCV), tetrahydrocannabinol (THC), cannabinol (CBN) or any combination thereof.

16. The oral cannabinoid compositions of embodiment 15, wherein the cannabinoid is cannabidiol (CBD).

17. The oral cannabinoid compositions of embodiment 15, wherein the cannabinoid is a combination of cannabidiol (CBD) and tetrahydrocannabinol (THC).

18. The oral cannabinoid compositions of any one of the embodiments 1 to 17, which comprises about 20% (w/w) of the solubilizing agent.

19. The oral cannabinoid compositions of any one of the embodiments 1 to 17, which comprises about 21% (w/w) of the solubilizing agent.

20 The oral cannabinoid compositions of any one of the embodiments 1 to 17, which comprises about 22% (w/w) of the solubilizing agent.

21. The oral cannabinoid compositions of any one of the embodiments 1 to 17, which comprises about 23% (w/w) of the solubilizing agent.

22. The oral cannabinoid compositions of any one of the embodiments 1 to 17, which comprises about 24% (w/w) of the solubilizing agent.

23. The oral cannabinoid compositions of any one of the embodiments 1 to 17, which comprises about 25% (w/w) of the solubilizing agent.

24. The oral cannabinoid compositions of any one of the embodiments 1 to 17, which comprises about 26% (w/w) of the solubilizing agent.

25 The oral cannabinoid compositions of any one of the embodiments 1 to 17, which comprises about 27% (w/w) of the solubilizing agent.

26. The oral cannabinoid compositions of any one of the embodiments 1 to 17, which comprises about 28% (w/w) of the solubilizing agent.

27. The oral cannabinoid compositions of any one of the embodiments 1 to 17, which comprises about 29% (w/w) of the solubilizing agent.

28. The oral cannabinoid compositions of any one of the embodiments 1 to 17, which comprises about 30% (w/w) of the solubilizing agent.

29. The oral cannabinoid compositions of any one of the embodiments 1 to 17, which comprises about 31% (w/w) of the solubilizing agent.

30. The oral cannabinoid compositions of any one of the embodiments 1 to 17, which comprises about 32% (w/w) of the solubilizing agent.

31. The oral cannabinoid compositions of any one of the embodiments 1 to 17, which comprises about 33% (w/w) of the solubilizing agent.

32. The oral cannabinoid compositions of any one of the embodiments 1 to 17, which comprises about 34% (w/w) of the solubilizing agent.

33. The oral cannabinoid compositions of any one of the embodiments 1 to 17, which comprises about 35% (w/w) of the solubilizing agent.

34. The oral cannabinoid compositions of any one of the embodiments 1 to 17, which comprises about 36% (w/w) of the solubilizing agent.

35. The oral cannabinoid compositions of any one of the embodiments 1 to 17, which comprises about 37% (w/w) of the solubilizing agent.

36. The oral cannabinoid compositions of any one of the embodiments 1 to 17, which comprises about 38% (w/w) of the solubilizing agent.

37. The oral cannabinoid compositions of any one of the embodiments 1 to 17, which comprises about 39% (w/w) of the solubilizing agent.

38. The oral cannabinoid compositions of any one of the embodiments 1 to 17, which comprises about 40% (w/w) of the solubilizing agent.

39. The oral cannabinoid compositions of any one of the embodiments 1 to 38, wherein the solubilizing agent is polyoxyl castor oil, Poloxamer, polysorbates, benzalkonium chloride, cyclodextrins, lecithin, benzyl alcohol, benzyl benzoate, or any combination thereof.

40. The oral cannabinoid compositions of embodiment 39, wherein the solubilizing agent is polyoxyl castor oil.

41. The oral cannabinoid compositions of any one of the embodiments 1 to 40, which comprises about 0.01% (w/w) of stabilizer.

42. The oral cannabinoid compositions of any one of the embodiments 1 to 40, which comprises about 0.1% (w/w) of stabilizer.

43. The oral cannabinoid compositions of any one of the embodiments 1 to 40, which comprises about 0.5% (w/w) of stabilizer.

44. The oral cannabinoid compositions of any one of the embodiments 1 to 40, which comprises about 1% (w/w) of stabilizer.

45. The oral cannabinoid compositions of any one of the embodiments 1 to 40, which comprises about 2% (w/w) of stabilizer.

46. The oral cannabinoid compositions of any one of the embodiments 1 to 40, which comprises about 3% (w/w) of stabilizer.

47. The oral cannabinoid compositions of any one of the embodiments 1 to 40, which comprises about 4% (w/w) of stabilizer.

48. The oral cannabinoid compositions of any one of the embodiments 1 to 41, which comprises about 5% (w/w) of stabilizer.

49. The oral cannabinoid compositions of any one of the embodiments 1 to 48, wherein the stabilizer is one or more organic acids, carboxylic acids, acid salts of amino acids such as cysteine hydrochloride, glycine hydrochloride, cystine dihydrochloride, ascorbic acid, malic acid, isoascorbic acid, citric acid, tartaric acid, palmityl, stearyl ascorbate, and mixtures thereof.

50. The oral cannabinoid compositions of embodiment 49, wherein the stabilizer is citric acid.

51. The oral cannabinoid compositions of any one of the embodiments 1 to 50, wherein the compositions are formulated as liquid, solid or powdered forms.

55. The oral liquid cannabinoid compositions of any one of the embodiments 1 to 51, which comprises about 1% (w/w) of oil or semi-solid fat.

56. The oral liquid cannabinoid compositions of any one of the embodiments 1 to 51, which comprises about 2% (w/w) of oil or semi-solid fat.

57. The oral liquid cannabinoid compositions of any one of the embodiments 1 to 51, which comprises about 3% (w/w) of oil or semi-solid fat.

58. The oral liquid cannabinoid compositions of any one of the embodiments 1 to 51, which comprises about 4% (w/w) of oil or semi-solid fat.

59. The oral liquid cannabinoid compositions of any one of the embodiments 1 to 51, which comprises about 5% (w/w) of oil or semi-solid fat.

60. The oral liquid cannabinoid compositions of any one of the embodiments 1 to 51, which comprises about 6% (w/w) of oil or semi-solid fat.

61. The oral liquid cannabinoid compositions of any one of the embodiments 1 to 51, which comprises about 7% (w/w) of oil or semi-solid fat.

62. The oral liquid cannabinoid compositions of any one of the embodiments 1 to 51, which comprises about 8% (w/w) of oil or semi-solid fat.

63. The oral liquid cannabinoid compositions of any one of the embodiments 1 to 51, which comprises about 9% (w/w) of oil or semi-solid fat.

64. The oral liquid cannabinoid compositions of any one of the embodiments 1 to 51, which comprises about 10% (w/w) of oil or semi-solid fat.

65. The oral liquid cannabinoid compositions of any one of the embodiments 1 to 51, which comprises about 11% (w/w) of oil or semi-solid fat.

66. The oral liquid cannabinoid compositions of any one of the embodiments 1 to 51, which comprises about 12% (w/w) of oil or semi-solid fat.

67. The oral liquid cannabinoid compositions of any one of the embodiments 1 to 51, which comprises about 13% (w/w) of oil or semi-solid fat.

68. The oral liquid cannabinoid compositions of any one of the embodiments 1 to 51, which comprises about 14% (w/w) of oil or semi-solid fat.

69. The oral liquid cannabinoid compositions of any one of the embodiments 1 to 51, which comprises about 15% (w/w) of oil or semi-solid fat.

70. The oral liquid cannabinoid compositions of any one of the embodiments 1 to 51, which comprises about 16% (w/w) of oil or semi-solid fat.

71. The oral liquid cannabinoid compositions of any one of the embodiments 1 to 51, which comprises about 17% (w/w) of oil or semi-solid fat.

72. The oral liquid cannabinoid compositions of any one of the embodiments 1 to 51, which comprises about 18% (w/w) of oil or semi-solid fat.

73. The oral liquid cannabinoid compositions of any one of the embodiments 1 to 51, which comprises about 19% (w/w) of oil or semi-solid fat.

74. The oral liquid cannabinoid compositions of any one of the embodiments 1 to 51, which comprises about 20% (w/w) of oil or semi-solid fat.

75. The oral liquid cannabinoid compositions of any one of the embodiments 1 to 74, wherein the oil or semi-solid fat comprises a variety of different oils e.g., soybean oil, olive oil, fish oil, fish oil extract, safflower oil, corn oil, sunflower oil, coconut oil, palm kernel oil, rapeseed oil, sesame oil, vegetable oils, mineral oils, medium chain triglycerides (MCT), and mixtures thereof.

76. The oral liquid cannabinoid compositions of embodiment 75, wherein the oil or semi-solid fat is medium chain triglyceride (MCT) oil extracted from coconut oil.

77. The oral liquid cannabinoid compositions of any one of the embodiments 1 to 76, which comprises a solvent to make 100% by weight of the composition.

78. The oral liquid cannabinoid compositions of any one of the embodiments 1 to 77, wherein the solvent is selected from the group consisting of diethylene glycol ethyl ether, methoxy polyethylene glycol, propylene glycol, polypropylene glycol, polyethylene glycol (PEG), glycerol, ethanol, dimethyl isosorbide, glycofurol, propylene carbonate, dimethyl acetamide, or mixtures thereof.

79. The oral liquid cannabinoid compositions of embodiment 78, wherein the solvent is propylene glycol.

80. The oral solid cannabinoid compositions of any one of the embodiments 1 to 51, which comprises about 1% (w/w) of surfactant.

81. The oral solid cannabinoid compositions of any one of the embodiments 1 to 51, which comprises about 2% (w/w) of surfactant.

82. The oral solid cannabinoid compositions of any one of the embodiments 1 to 51, which comprises about 3% (w/w) of surfactant.

83. The oral solid cannabinoid compositions of any one of the embodiments 1 to 51, which comprises about 4% (w/w) of surfactant.

84. The oral solid cannabinoid compositions of any one of the embodiments 1 to 51, which comprises about 5% (w/w) of surfactant.

85. The oral solid cannabinoid compositions of any one of the embodiments 1 to 51, which comprises about 6% (w/w) of surfactant.

86. The oral solid cannabinoid compositions of any one of the embodiments 1 to 51, which comprises about 7% (w/w) of surfactant.

87. The oral solid cannabinoid compositions of any one of the embodiments 1 to 51, which comprises about 8% (w/w) of surfactant.

88. The oral solid cannabinoid compositions of any one of the embodiments 1 to 51, which comprises about 9% (w/w) of surfactant.

89. The oral solid cannabinoid compositions of any one of the embodiments 1 to 51, which comprises about 10% (w/w) of surfactant.

90. The oral solid cannabinoid compositions of any one of the embodiments 1 to 51, which comprises about 11% (w/w) of surfactant.

91. The oral solid cannabinoid compositions of any one of the embodiments 1 to 51, which comprises about 12% (w/w) of surfactant.

92. The oral solid cannabinoid compositions of any one of the embodiments 1 to 51, which comprises about 13% (w/w) of surfactant.

93. The oral solid cannabinoid compositions of any one of the embodiments 1 to 51, which comprises about 14% (w/w) of surfactant.

94 The oral solid cannabinoid compositions of any one of the embodiments 1 to 51, which comprises about 15% (w/w) of surfactant.

95 The oral solid cannabinoid compositions of any one of the embodiments 1 to 51 and 80 to 94, wherein the surfactant is a non-ionic surfactant such as polysorbate 20 (Tween 20), polysorbate 40 (Tween 40), polysorbate 60 (Tween 60), polysorbate 80 (Tween 80), Poloxamer, and mixtures thereof.

96. The oral solid cannabinoid compositions of embodiment 95, wherein the surfactant is Poloxamer.

97. The oral solid cannabinoid compositions of any one of the embodiments 1 to 51 and 80 to 96, which comprises about 1% (w/w) of oil or semi-solid fat.

98. The oral solid cannabinoid compositions of any one of the embodiments 1 to 51 and 80 to 96, which comprises about 2% (w/w) of oil or semi-solid fat.

99 The oral solid cannabinoid compositions of any one of the embodiments 1 to 51 and 80 to 96, which comprises about 3% (w/w) of oil or semi-solid fat.

100. The oral solid cannabinoid compositions of any one of the embodiments 1 to 51 and 80 to 96, which comprises about 4% (w/w) of oil or semi-solid fat.

101. The oral solid cannabinoid compositions of any one of the embodiments 1 to 51 and 80 to 96, which comprises about 5% (w/w) of oil or semi-solid fat.

102. The oral solid cannabinoid compositions of any one of the embodiments 1 to 51 and 80 to 96, which comprises about 6% (w/w) of oil or semi-solid fat.

103. The oral solid cannabinoid compositions of any one of the embodiments 1 to 51 and 80 to 96, which comprises about 7% (w/w) of oil or semi-solid fat.

104. The oral solid cannabinoid compositions of any one of the embodiments 1 to 51 and 80 to 96, which comprises about 8% (w/w) of oil or semi-solid fat.

105. The oral solid cannabinoid compositions of any one of the embodiments 1 to 51 and 80 to 96, which comprises about 9% (w/w) of oil or semi-solid fat.

106. The oral solid cannabinoid compositions of any one of the embodiments 1 to 51 and 80 to 96, which comprises about 10% (w/w) of oil or semi-solid fat.

107.

The oral solid cannabinoid compositions of any one of the embodiments 1 to 51 and 80 to 96, which comprises about 11% (w/w) of oil or semi-solid fat.

108. The oral solid cannabinoid compositions of any one of the embodiments 1 to 51 and 80 to 96, which comprises about 12% (w/w) of oil or semi-solid fat.

109. The oral solid cannabinoid compositions of any one of the embodiments 1 to 51 and 80 to 96, which comprises about 13% (w/w) of oil or semi-solid fat.

110. The oral solid cannabinoid compositions of any one of the embodiments 1 to 51 and 80 to 96, which comprises about 14% (w/w) of oil or semi-solid fat.

111. The oral solid cannabinoid compositions of any one of the embodiments 1 to 51 and 80 to 96, which comprises about 15% (w/w) of oil or semi-solid fat.

112. The oral solid cannabinoid compositions of any one of the embodiments 1 to 51 and 80 to 96, which comprises about 16% (w/w) of oil or semi-solid fat.

113. The oral solid cannabinoid compositions of any one of the embodiments 1 to 51 and 80 to 96, which comprises about 17% (w/w) of oil or semi-solid fat.

114. The oral solid cannabinoid compositions of any one of the embodiments 1 to 51 and 80 to 96, which comprises about 18% (w/w) of oil or semi-solid fat.

115. The oral solid cannabinoid compositions of any one of the embodiments 1 to 51 and 80 to 96, which comprises about 19% (w/w) of oil or semi-solid fat.

116. The oral solid cannabinoid compositions of any one of the embodiments 1 to 51 and 80 to 96, which comprises about 20% (w/w) of oil or semi-solid fat.

117. The oral solid cannabinoid compositions of any one of the embodiments 1 to 51 and 80 to 117, wherein the oil or semi-solid fat comprises a variety of different oils e.g., soybean oil, olive oil, fish oil, fish oil extract, safflower oil, corn oil, sunflower oil, coconut oil, palm kernel oil, rapeseed oil, sesame oil, vegetable oils, mineral oils, medium chain triglycerides (MCT), and mixtures thereof.

118. The oral solid cannabinoid compositions of embodiment 117, wherein the oil or semi-solid fat is medium chain triglyceride (MCT) oil extracted from coconut oil.

119. The oral solid cannabinoid compositions of any one of the embodiments 1 to 51 and 80 to 119, which comprises about 1% (w/w) of a solvent.

120. The oral solid cannabinoid compositions of any one of the embodiments 1 to 51 and 80 to 119, which comprises about 2% (w/w) of a solvent.

121. The oral solid cannabinoid compositions of any one of the embodiments 1 to 51 and 80 to 119, which comprises about 3% (w/w) of a solvent.

122. The oral solid cannabinoid compositions of any one of the embodiments 1 to 51 and 80 to 119, which comprises about 4% (w/w) of a solvent.

123. The oral solid cannabinoid compositions of any one of the embodiments 1 to 51 and 80 to 119, which comprises about 5% (w/w) of a solvent.

124. The oral solid cannabinoid compositions of any one of the embodiments 1 to 51 and 80 to 123, wherein the solvent is selected from the group consisting of diethylene glycol ethyl ether, methoxy polyethylene glycol, propylene glycol, polypropylene glycol, polyethylene glycol (PEG), glycerol, ethanol, dimethyl isosorbide, glycofurol, propylene carbonate, dimethyl acetamide, mixtures thereof.

125. The oral solid cannabinoid compositions of embodiment 124, wherein the solvent is propylene glycol.

126. The oral solid cannabinoid compositions of any one of the embodiments 1 to 51 and 80 to 125, which comprises a co-solvent to make 100% by weight of the composition.

127. The oral solid cannabinoid compositions of any one of the embodiments 1 to 51 and 80 to 126, wherein the co-solvent is selected from the group consisting of diethylene glycol ethyl ether, methoxy polyethylene glycol, propylene glycol, polypropylene glycol, polyethylene glycol (PEG), glycerol, ethanol, dimethyl isosorbide, glycofurol, propylene carbonate, dimethyl acetamide, mixtures thereof.

128. The oral solid cannabinoid compositions of embodiment 127, wherein the co-solvent is polyethylene glycol.

129. The oral powdered cannabinoid compositions of any one of the embodiments 1 to 51, which comprises about 20% (w/w) of surfactant.

130. The oral powdered cannabinoid compositions of any one of the embodiments 1 to 51, which comprises about 21% (w/w) of surfactant.

131. The oral powdered cannabinoid compositions of any one of the embodiments 1 to 51, which comprises about 22% (w/w) of surfactant.

132. The oral powdered cannabinoid compositions of any one of the embodiments 1 to 51, which comprises about 23% (w/w) of surfactant.

133. The oral powdered cannabinoid compositions of any one of the embodiments 1 to 51, which comprises about 24% (w/w) of surfactant.

134. The oral powdered cannabinoid compositions of any one of the embodiments 1 to 51, which comprises about 25% (w/w) of surfactant.

135. The oral powdered cannabinoid compositions of any one of the embodiments 1 to 51, which comprises about 26% (w/w) of surfactant.

136. The oral powdered cannabinoid compositions of any one of the embodiments 1 to 51, which comprises about 27% (w/w) of surfactant.

137. The oral powdered cannabinoid compositions of any one of the embodiments 1 to 51, which comprises about 28% (w/w) of surfactant.

138. The oral powdered cannabinoid compositions of any one of the embodiments 1 to 51, which comprises about 29% (w/w) of surfactant.

139. The oral powdered cannabinoid compositions of any one of the embodiments 1 to 51, which comprises about 30% (w/w) of surfactant.

140. The oral powdered cannabinoid compositions of any one of the embodiments 1 to 51, which comprises about 31% (w/w) of surfactant.

141. The oral powdered cannabinoid compositions of any one of the embodiments 1 to 51, which comprises about 32% (w/w) of surfactant.

142. The oral powdered cannabinoid compositions of any one of the embodiments 1 to 51, which comprises about 33% (w/w) of surfactant.

143. The oral powdered cannabinoid compositions of any one of the embodiments 1 to 51, which comprises about 34% (w/w) of surfactant.

144. The oral powdered cannabinoid compositions of any one of the embodiments 1 to 51, which comprises about 35% (w/w) of surfactant.

145. The oral powdered cannabinoid compositions of any one of the embodiments 1 to 51, which comprises about 36% (w/w) of surfactant.

146. The oral powdered cannabinoid compositions of any one of the embodiments 1 to 51, which comprises about 37% (w/w) of surfactant.

147. The oral powdered cannabinoid compositions of any one of the embodiments 1 to 51, which comprises about 38% (w/w) of surfactant.

148. The oral powdered cannabinoid compositions of any one of the embodiments 1 to 51, which comprises about 39% (w/w) of surfactant.

149. The oral powdered cannabinoid compositions of any one of the embodiments 1 to 51, which comprises about 40% (w/w) of surfactant.

150. The oral powdered cannabinoid compositions of any one of the embodiments 1 to 51 and 129 to 150, wherein the surfactant is a non-ionic surfactant including polysorbate 20 (Tween 20), polysorbate 40 (Tween 40), polysorbate 60 (Tween 60), polysorbate 80 (Tween 80), Poloxamer, and mixtures thereof.

151. The oral powdered cannabinoid compositions of embodiment 150, wherein the surfactant is Poloxamer.

152. The oral powdered cannabinoid compositions of any one of the embodiments 1 to 51 and 129 to 151, which comprises about 1% (w/w) of a solvent.

153. The oral powdered cannabinoid compositions of any one of the embodiments 1 to 51 and 129 to 151, which comprises about 2% (w/w) of a solvent.

154. The oral powdered cannabinoid compositions of any one of the embodiments 1 to 51 and 129 to 151, which comprises about 3% (w/w) of a solvent.

155. The oral powdered cannabinoid compositions of any one of the embodiments 1 to 51 and 129 to 151, which comprises about 4% (w/w) of a solvent.

156. The oral powdered cannabinoid compositions of any one of the embodiments 1 to 51 and 129 to 151, which comprises about 5% (w/w) of a solvent.

157. The oral powdered cannabinoid compositions of any one of the embodiments 1 to 51 and 129 to 156, wherein the solvent is selected from the group consisting of diethylene glycol ethyl ether, methoxy polyethylene glycol, propylene glycol, polypropylene glycol, polyethylene glycol (PEG), glycerol, ethanol, dimethyl isosorbide, glycofurol, propylene carbonate, dimethyl acetamide, or mixtures thereof.

158. The oral powdered cannabinoid compositions of embodiment 157, wherein the solvent is propylene glycol.

159. The oral powdered cannabinoid compositions of any one of the embodiments 1 to 51 and 129 to 158, which comprises about 20% (w/w) of a thickening agent.

160. The oral powdered cannabinoid compositions of any one of the embodiments 1 to 51 and 129 to 158, which comprises about 21% (w/w) of a thickening agent.

161. The oral powdered cannabinoid compositions of any one of the embodiments 1 to 51 and 129 to 158, which comprises about 22% (w/w) of a thickening agent.

162. The oral powdered cannabinoid compositions of any one of the embodiments 1 to 51 and 129 to 158, which comprises about 23% (w/w) of a thickening agent.

163. The oral powdered cannabinoid compositions of any one of the embodiments 1 to 51 and 129 to 158, which comprises about 24% (w/w) of a thickening agent.

164. The oral powdered cannabinoid compositions of any one of the embodiments 1 to 51 and 129 to 158, which comprises about 25% (w/w) of a thickening agent.

165. The oral powdered cannabinoid compositions of any one of the embodiments 1 to 51 and 129 to 158, which comprises about 26% (w/w) of a thickening agent.

166. The oral powdered cannabinoid compositions of any one of the embodiments 1 to 51 and 129 to 158, which comprises about 27% (w/w) of a thickening agent.

167. The oral powdered cannabinoid compositions of any one of the embodiments 1 to 51 and 129 to 158, which comprises about 28% (w/w) of a thickening agent.

168. The oral powdered cannabinoid compositions of any one of the embodiments 1 to 51 and 129 to 158, which comprises about 29% (w/w) of a thickening agent.

169. The oral powdered cannabinoid compositions of any one of the embodiments 1 to 51 and 129 to 158, which comprises about 30% (w/w) of a thickening agent.

170. The oral powdered cannabinoid compositions of any one of the embodiments 1 to 51 and 129 to 169, wherein the thickening agent is selected from the group consisting of bentones, gums, silicon dioxide, stearyl alcohol, castor wax, or mixtures thereof.

171. The oral powdered cannabinoid compositions of embodiment 170, wherein the thickening agent is silicon dioxide.

172. The oral powdered cannabinoid compositions of any one of the embodiments 1 to 51 and 129 to 171, wherein the powdered composition is used to formulate controlled release tablets using a standardized matrix by melt granulation process.

173. The controlled release tablet cannabinoid compositions of embodiment 172, which comprises about 20% (w/w) of a carrier.

174. The controlled release tablet cannabinoid compositions of embodiment 172, which comprises about 21% (w/w) of a carrier.

175. The controlled release tablet cannabinoid compositions of embodiment 172, which comprises about 22% (w/w) of a carrier.

176. The controlled release tablet cannabinoid compositions of embodiment 172, which comprises about 23% (w/w) of a carrier.

177. The controlled release tablet cannabinoid compositions of embodiment 172, which comprises about 24% (w/w) of a carrier.

178. The controlled release tablet cannabinoid compositions of embodiment 172, which comprises about 25% (w/w) of a carrier.

179. The controlled release tablet cannabinoid compositions of embodiment 172, which comprises about 26% (w/w) of a carrier.

180. The controlled release tablet cannabinoid compositions of embodiment 172, which comprises about 27% (w/w) of a carrier.

181. The controlled release tablet cannabinoid compositions of embodiment 172, which comprises about 28% (w/w) of a carrier.

182. The controlled release tablet cannabinoid compositions of embodiment 172, which comprises about 29% (w/w) of a carrier.

183. The controlled release tablet cannabinoid compositions of embodiment 172, which comprises about 30% (w/w) of a carrier.

184. The controlled release tablet cannabinoid compositions of embodiment 172, which comprises about 31% (w/w) of a carrier.

185. The controlled release tablet cannabinoid compositions of embodiment 172, which comprises about 32% (w/w) of a carrier.

186. The controlled release tablet cannabinoid compositions of embodiment 172, which comprises about 33% (w/w) of a carrier.

187. The controlled release tablet cannabinoid compositions of embodiment 172, which comprises about 34% (w/w) of a carrier.

188. The controlled release tablet cannabinoid compositions of embodiment 172, which comprises about 35% (w/w) of a carrier.

189. The controlled release tablet cannabinoid compositions of embodiment 172, which comprises about 36% (w/w) of a carrier.

190. The controlled release tablet cannabinoid compositions of embodiment 172, which comprises about 37% (w/w) of a carrier.

191. The controlled release tablet cannabinoid compositions of embodiment 172, which comprises about 38% (w/w) of a carrier.

192. The controlled release tablet cannabinoid compositions of embodiment 172, which comprises about 39% (w/w) of a carrier.

193. The controlled release tablet cannabinoid compositions of embodiment 172, which comprises about 40% (w/w) of a carrier.

194. The controlled release tablet cannabinoid compositions of any one of the embodiments 172 to 194, wherein the carrier is selected from the group consisting of bentones, gums, silicon dioxide, stearyl alcohol, castor wax, or mixtures thereof.

195. The controlled release tablet cannabinoid compositions of embodiment 194, wherein the carrier is silicon dioxide.

196. The controlled release tablet cannabinoid compositions of embodiments 172 to 195, which comprises about 1% (w/w) of a gelling agent.

197. The controlled release tablet cannabinoid compositions of embodiments 172 to 195, which comprises about 2% (w/w) of a gelling agent.

198. The controlled release tablet cannabinoid compositions of embodiments 172 to 195, which comprises about 3% (w/w) of a gelling agent.

199. The controlled release tablet cannabinoid compositions of embodiments 172 to 195, which comprises about 4% (w/w) of a gelling agent.

200. The controlled release tablet cannabinoid compositions of embodiments 172 to 195, which comprises about 5% (w/w) of a gelling agent.

201. The controlled release tablet cannabinoid compositions of embodiments 172 to 195, which comprises about 6% (w/w) of a gelling agent.

202. The controlled release tablet cannabinoid compositions of embodiments 172 to 195, which comprises about 7% (w/w) of a gelling agent.

203. The controlled release tablet cannabinoid compositions of embodiments 172 to 195, which comprises about 8% (w/w) of a gelling agent.

204. The controlled release tablet cannabinoid compositions of embodiments 172 to 195, which comprises about 9% (w/w) of a gelling agent.

205. The controlled release tablet cannabinoid compositions of embodiments 172 to 195, which comprises about 10% (w/w) of a gelling agent.

206. The controlled release tablet cannabinoid compositions of embodiments 172 to 205, wherein the gelling agent is selected from the group consisting of the family of polyacrylamides; copolymers of acrylic acid; “electrolyte-insensitive” carbomers; polysaccharides; cellulose and derivatives thereof; and magnesium aluminum silicates. Examples include sodium acryloyldimethyltaurate copolymer/isohexadecane/polysorbate 80 mixture, the polyacrylamide/C13-14 isoparaffin/laureth-7 mixture, Carbopol 1382, Carbopol 974, Carbopol 980, Pemulen™ TR, xanthan gum, hydroxypropylmethylcellulose or hydroxyethylcellulose, combinations, and mixtures thereof.

207. The controlled release tablet cannabinoid compositions of embodiment 206, wherein the gelling agent is Carbopol 974.

208. The controlled release tablet cannabinoid compositions of embodiments 172 to 206, which comprises about 1% (w/w) of a plasticizer.

209. The controlled release tablet cannabinoid compositions of embodiments 172 to 206, which comprises about 2% (w/w) of a plasticizer.

210. The controlled release tablet cannabinoid compositions of embodiments 172 to 206, which comprises about 3% (w/w) of a plasticizer.

211. The controlled release tablet cannabinoid compositions of embodiments 172 to 206, which comprises about 4% (w/w) of a plasticizer.

212. The controlled release tablet cannabinoid compositions of embodiments 172 to 206, which comprises about 5% (w/w) of a plasticizer.

213. The controlled release tablet cannabinoid compositions of embodiments 172 to 206, which comprises about 6% (w/w) of a plasticizer.

214. The controlled release tablet cannabinoid compositions of embodiments 172 to 206, which comprises about 7% (w/w) of a plasticizer.

215. The controlled release tablet cannabinoid compositions of embodiments 172 to 206, which comprises about 8% (w/w) of a plasticizer.

216. The controlled release tablet cannabinoid compositions of embodiments 172 to 206, which comprises about 9% (w/w) of a plasticizer.

217. The controlled release tablet cannabinoid compositions of embodiments 172 to 206, which comprises about 10% (w/w) of a plasticizer.

218. The controlled release tablet cannabinoid compositions of embodiments 172 to 217, wherein the plasticizer is a polymer such as hydroxypropyl-methylcellulose (HPMC), polyvinylpyrrolidone (PVP), or mixtures thereof.

219. The controlled release tablet cannabinoid compositions of embodiment 218, wherein the plasticizer is polyvinylpyrrolidone (PVP).

220. The controlled release tablet cannabinoid compositions of embodiments 172 to 219, which comprises about 1% (w/w) of flavoring agent.

221. The controlled release tablet cannabinoid compositions of embodiments 172 to 219, which comprises about 2% (w/w) of flavoring agent.

222. The controlled release tablet cannabinoid compositions of embodiments 172 to 219, which comprises about 3% (w/w) of flavoring agent.

223. The controlled release tablet cannabinoid compositions of embodiments 172 to 219, which comprises about 4% (w/w) of flavoring agent.

224. The controlled release tablet cannabinoid compositions of embodiments 172 to 219, which comprises about 5% (w/w) of flavoring agent.

225. The controlled release tablet cannabinoid compositions of embodiments 172 to 219, which comprises about 6% (w/w) of flavoring agent.

226. The controlled release tablet cannabinoid compositions of embodiments 172 to 219, which comprises about 7% (w/w) of flavoring agent.

227. The controlled release tablet cannabinoid compositions of embodiments 172 to 219, which comprises about 8% (w/w) of flavoring agent.

228. The controlled release tablet cannabinoid compositions of embodiments 172 to 219, which comprises about 9% (w/w) of flavoring agent.

229. The controlled release tablet cannabinoid compositions of embodiments 172 to 219, which comprises about 10% (w/w) of flavoring agent.

230. The controlled release tablet cannabinoid compositions of embodiments 172 to 229, wherein the flavoring agent is caraway, clove, lemon, spearmint, rose, peppermint, ginger, raspberry, maltol, syrups, cherry, or mixtures thereof.

231. The controlled release tablet cannabinoid compositions of embodiment 230, wherein the flavoring agent is spearmint.

232. The controlled release tablet cannabinoid compositions of embodiments 172 to 231, which comprises about 1% (w/w) of lubricant.

233. The controlled release tablet cannabinoid compositions of embodiments 172 to 231, which comprises about 2% (w/w) of lubricant.

234. The controlled release tablet cannabinoid compositions of embodiments 172 to 233, wherein the lubricant is selected from metallic salts of fatty acids such as magnesium stearate, calcium stearate, and zinc stearate; or fatty acids such as stearic acid; or fatty acid esters, such as glyceride esters (glyceryl monostearate, glyceryl tribehenate, and glyceryl dibehenate) and sugar esters (sorbitan monostearate and sucrose monopalmitate), or mixtures thereof.

235. The controlled release tablet cannabinoid compositions of embodiment 234, wherein the lubricant is magnesium stearate.

236. The controlled release tablet cannabinoid compositions of embodiments 172 to 235, which comprises filler to make 100% by weight of the composition.

237. The controlled release tablet cannabinoid compositions of embodiments 172 to 236, wherein the filler is gelatin, microcrystalline cellulose powder, carrageenan, titanium dioxide, or mixtures thereof.

238. The controlled release tablet cannabinoid compositions of embodiment 237, wherein the filler is microcrystalline cellulose powder.

239. The oral cannabinoid compositions of any one of the embodiments from 1 to 238, wherein the compositions are emulsion pre-concentrates at the time of administration.

240. The oral cannabinoid compositions of embodiment 239, wherein the emulsion pre-concentrates are filled into single unit dosage forms such as capsules, drinking ampoules and dose cushions, or alternatively be formed as other suitable dosage forms such as powder, granules, pellets, tablets, chewable soft pills and chewy-base lozenges.

241. An oral liquid cannabinoid composition comprising:
a. a cannabinoid at 0.01-10% (w/w),
b. a solubilizing agent at 20-40% (w/w),
c. a stabilizer at 0.01-5% (w/w),
d. an oil or semi-solid fat at 1-20% (w/w), and
e. a solvent to make 100% by weight.
wherein the liquid composition is soluble in water.

242. An oral capsule cannabinoid composition comprising:
a. a cannabinoid at 0.01-10% (w/w),
b. a solubilizing agent at 20-40% (w/w),
c. a stabilizer at 0.01-5% (w/w),
d. a surfactant at 1-15% (w/w),
e. an oil or semi-solid fat at 1-20% (w/w),
f. a solvent at 1-5% (w/w), and
g. a co-solvent to make 100% by weight.

243. An oral powdered cannabinoid composition comprising:
a. a cannabinoid at 0.01-10% (w/w),
b. a solubilizing agent at 20-40% (w/w),
c. a stabilizer at 0.01-5% (w/w),
d. a surfactant at 20-40% (w/w),
e. a solvent at 1-5% (w/w), and
f. a thickening agent at 20-30% (w/w).

244. An oral controlled release tablet composition comprising:
a. a cannabinoid at 0.01-10% (w/w),
b. a solubilizing agent at 20-40% (w/w),
c. a stabilizer at 0.01-5% (w/w),
d. a surfactant at 20-40% (w/w),
e. a solvent at 1-5% (w/w), and
f. a thickening agent or carrier at 20-30% (w/w),
g. a gelling agent at 1-10% (w/w),
h. a plasticizer at 1-10% (w/w),
i. a flavoring agent at 1-10% (w/w),
j. a lubricant at 1-2% (w/w), and
k. a filler to make 100% by weight.

245. The oral cannabinoid compositions of any one of the embodiments 241 to 244, wherein the compositions are self-emulsifying and form in-situ emulsion upon contact with aqueous media such as gastrointestinal fluids.

246. The method comprising administering the oral cannabinoid compositions of any one of embodiments 1 to 245 to a subject for the treatment of neurological diseases and/or disorders.

247. The method of embodiment 246, wherein the neurological diseases and disorders include but are not limited to headaches; depression; anxiety; epileptic seizures; disorders; dementias; sleep disorders; Amyotrophic Lateral Sclerosis (ALS); stroke; Parkinson's Disease; neuropathic pain due to amputation; cancer; carpal tunnel syndrome; chemotherapy; diabetes; facial nerve problems; HIV infection/AIDS; multiple myeloma; multiple sclerosis; nerve or spinal cord compression from herniated discs or from arthritis in the spine; shingles; spine surgery; syphilis; thyroid problems or vitamin B deficiency.

248. The method of any one of embodiments 246 or 247, wherein the subject is a mammal.

249. The method of any one of embodiments 246 to 248, wherein the subject is a companion animal.

250. The method of embodiment 248, wherein the subject is a human.

251. The method of any one of embodiments 246 to 250, wherein the method further comprises administering an additional therapy for neurological disease and/or disorder to a subject.

252. Use of the oral cannabinoid compositions of any one of the embodiments 1 to 245 for the treatment of neurological disease and/or disorder in a subject.

253. The use of embodiment 252, wherein the neurological disease and/or disorder include but are not limited to headaches; depression; anxiety; epileptic seizures; movement disorders; dementias; sleep disorders; Amyotrophic Lateral Sclerosis (ALS); stroke; Parkinson's Disease; neuropathic pain due to amputation; cancer; carpal tunnel syndrome; chemotherapy; diabetes; facial nerve problems; HIV infection/AIDS; multiple myeloma; multiple sclerosis; nerve or spinal cord compression from herniated discs or from arthritis in the spine; shingles; spine surgery; syphilis; thyroid problems or vitamin B deficiency.

254. The use of any one of embodiments 252 or 253, wherein the subject is a mammal.

255. The use of any one of embodiments 252 to 254, wherein the subject is a companion animal.

256. The use of embodiment 254, wherein the subject is a human.

257. The use of any one of embodiments 252 to 256, wherein the treatment further comprises an additional therapy for neurological disease and/or disorder.

### NON-LIMITING EXAMPLES

The discussion herein and the following Examples set forth and illustrate various exemplary embodiments of the present disclosure, which are understood to be illustrative and non-limiting.

### Example 1: Liquid Oral Cannabinoid SEDDS Composition

TABLE 1
Liquid cannabinoid composition

| Component | % w/w |
|---|---|
| CBD or THC | 2.5-10 |
| Polyoxyl castor oil | 30 |
| Propylene Glycol | 49.65 |
| MCT Oil | 0-20 |
| Citric Acid anhydrous | 0-0.2 |
| Total | 100 |

### Example 2: Solid Oral Cannabinoid SEDDS Composition

TABLE 2
Solid cannabinoid composition 1

| Component | % w/w |
|---|---|
| CBD | 3.12 |
| THC | 1.56 |
| Poloxamer | 12.5 |
| Polyoxyl castor oil | 25 |
| Propylene Glycol | 0.75 |
| Citric acid | 0.25 |
| Polyethylene glycol | 54.31 |
| MCT oil | 2.5 |
| Total | 100 |

TABLE 3
Solid cannabinoid composition 2

| Component | % w/w |
|---|---|
| CBD | 10 |
| Poloxamer | 12.5 |
| Polyoxyl castor oil | 25 |
| Propylene Glycol | 0.75 |
| Citric acid | 0.25 |
| Polyethylene glycol | 49 |
| MCT oil | 2.5 |
| Total | 100 |

TABLE 4
Solid cannabinoid composition 3

| Component | % w/w |
|---|---|
| CBD | 5 |
| Poloxamer | 12.5 |
| Polyoxyl castor oil | 25 |
| Propylene Glycol | 0.75 |
| Citric acid | 0.25 |
| Polyethylene glycol | 54 |
| MCT oil | 2.5 |
| Total | 100 |

### Example 2: Powdered Oral Cannabinoid SEDDS Composition

TABLE 5
Powdered cannabinoid composition

| Component | % w/w |
|---|---|
| CBD | 10 |
| Poloxamer | 35 |
| Polyoxyl castor oil | 25 |
| Propylene glycol | 2.4 |
| Citric acid | 0.6 |
| Silicon dioxide | 27 |
| Total | 100 |

Example 2: The powdered composition described in Table 5 was used to formulate controlled release tablet composition using a standardized matrix (Table 6) by wet granulation process to control the release of cannabinoids upon contact with aqueous media such as gastrointestinal fluids.

TABLE 6
Controlled release tablet matrix

| Component | % w/w |
|---|---|
| Silica | 30 |
| microcrystalline cellulose powder | 54.5 |
| Carbopol 974 | 5 |
| Spearmint flavour | 5 |
| Polyvinylpyrrolidone (PVP) | 5 |
| Magnesium stearate | 0.5 |
| Total | 100 |

While the foregoing has presented specific embodiments of the present disclosure, it is to be understood that these embodiments have been presented by way of example only. It is expected that others skilled in the art will perceive variations which, while varying from the foregoing, do not depart from the spirit and scope of the disclosure herein.

It must be noted that as used in this specification and the appended claims, the singular forms “a,” “an,” and “the” include plural reference unless the context clearly dictates otherwise. Unless defined otherwise all technical and scientific terms used herein have the same meaning as commonly understood to one of ordinary skill in the art to which this disclosure belongs.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to encompass the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items.

As used herein, whether in the specification or the appended claims, the transitional terms “comprising”, “including”, “having”, “containing”, “involving”, and the like are to be understood as being inclusive or open-ended (i.e., to mean including but not limited to), and they do not exclude unrecited elements, materials, or method steps. Only the transitional phrases “consisting of” and “consisting essentially of”, respectively, are closed or semi-closed transitional phrases with respect to claims and exemplary embodiment paragraphs herein. The transitional phrase “consisting of” excludes any element, step, or ingredient which is not specifically recited. The transitional phrase “consisting essentially of” limits the scope to the specified elements, materials, or steps and to those that do not materially affect the basic characteristic(s) of the disclosure herein.

1. Self-emulsifying oral compositions comprising a cannabinoid at 0.01-10% (w/w), a solubilizer at 20-40% (w/w) and a stabilizer at 0.01-5% (w/w), wherein the said compositions forms in-situ emulsion upon contact with aqueous media such as gastrointestinal fluids.

2. The oral cannabinoid compositions of claim 1, wherein the cannabinoid is cannabidiol (CBD), cannabidiolic acid (CBDA), cannabidivarin (CBDV), cannabigerol (CBG), cannabigervarin (CBGV), tetrahydrocannabivarin (THCV), tetrahydrocannabinol (THC), cannabinol (CBN) or any combination thereof.

3. The oral cannabinoid compositions of claim 2, wherein the cannabinoid is cannabidiol (CBD) or a combination of cannabidiol (CBD) and tetrahydrocannabinol (THC).

4. The oral cannabinoid compositions of claim 1, wherein the solubilizer is polyoxyl castor oil, Poloxamer, polysorbate, benzalkonium chloride, cyclodextrins, lecithin, benzyl alcohol, benzyl benzoate, and mixtures thereof.

5. The oral cannabinoid compositions of claim 4, wherein the solubilizer is polyoxyl castor oil.

6. The oral cannabinoid compositions of claim 1, wherein the stabilizer is organic acid, carboxylic acid, acid salt of amino acid such as cysteine hydrochloride, glycine hydrochloride, cystine dihydrochloride, ascorbic acid, malic acid, isoascorbic acid, citric acid, tartaric acid, palmityl, stearyl ascorbate, and mixtures thereof.

7. The oral cannabinoid compositions of claim 6, wherein the stabilizer is citric acid.

8. The oral cannabinoid compositions of claim 1, wherein the compositions are filled into single unit dosage forms such as self-emulsifying liquid, capsules, drinking ampoules, dose cushions, or any other suitable dosage forms such as powder, granules, pellets, tablets, chewable soft pills, and chewy-base lozenges.

9. The oral cannabinoid compositions of claim 8, wherein the composition is self-emulsifying liquid comprising an oil or semi solid fat 1-20% (w/w), and a solvent to make 100% by weight.

10. The oral cannabinoid compositions of claim 9, wherein the oil or semi solid fat is a medium chain triglyceride oil (MCT oil) extracted from coconut oil.

11. (canceled)

12. The oral cannabinoid compositions of claim 8, wherein the composition is a self-emulsifying solid capsule comprising a surfactant at 1-15% (w/w), an oil or semi-solid fat at 1-20% (w/w), a solvent at 1-5% (w/w), and a co-solvent to make 100% by weight.

13-16. (canceled)

17. The oral cannabinoid compositions of claim 8, wherein the composition is a self-emulsifying powder comprising a surfactant at 20-40% (w/w), a solvent at 1-5% (w/w), and a thickening agent at 20-30% (w/w).

18-19. (canceled)

20. The oral cannabinoid compositions of claim 17, wherein the thickening agent is silicon dioxide.

21. The oral cannabinoid compositions of claim 17, wherein the powdered composition is pressed to form a self-emulsifying tablet composition by melt granulation process.

22. The oral cannabinoid compositions of claim 21, wherein the tablet composition is a controlled release composition comprising a carrier at 20-40% (w/w), a gelling agent at 1-10% (w/w), a flavoring agent at 1-10% (w/w), a plasticizer at 1-10% (w/w), a lubricant at 1-2% (w/w), and a filler to make 100% by weight.

23. The oral cannabinoid compositions of claim 22, wherein the carrier is silicon dioxide.

24-25. (canceled)

26. The oral cannabinoid compositions of claim 22, wherein the plasticizer is polyvinylpyrrolidone (PVP).

27. The oral cannabinoid compositions of claim 22, wherein the lubricant is magnesium stearate.

28. The oral cannabinoid compositions of claim 22, wherein the filler is microcrystalline cellulose.

29. Use of the oral cannabinoid compositions of claim 1 for the treatment of neurological diseases and disorders including headaches; depression; anxiety; epileptic seizures; movement disorders isorders; dementias; sleep disorders; Amyotrophic Lateral Sclerosis (ALS); stroke; Parkinson's Disease; neuropathic pain due to amputation; cancer; carpal tunnel syndrome; chemotherapy; diabetes; facial nerve problems; HIV infection/AIDS; multiple myeloma; multiple sclerosis; nerve or spinal cord compression from herniated discs or from arthritis in the spine; shingles; spine surgery; syphilis; thyroid problems or vitamin B deficiency.
